(19) United States
(12) Patent Application Publication
(10) Pub. No.: US 2014/0330132 A1
(43) Pub. Date: Nov. 6, 2014

(54) PHYSIOLOGICAL CHARACTERISTIC DETECTION BASED ON REFLECTED COMPONENTS OF LIGHT

(71) Applicant: Aza Raskin, San Francisco, CA (US)

(72) Inventor: Aza Raskin, San Francisco, CA (US)

(21) Appl. No.: 13/886,254

(22) Filed: May 2, 2013

(51) Int. Cl. A61B 5/00; A61B 5/024

(52) U.S. Cl. CPC A61B 5/0059; A61B 5/02427; A61B 5/0077
USPC 600/479; 600/476

(57) ABSTRACT

Embodiments relate generally to the health field, and more specifically to a new and useful method for measuring detecting physiological characteristics, such as heart rate, of an organism. In one embodiment, a method includes detecting one or more surfaces associated with an organism, and receiving components of light reflected from the one or more surfaces of the organism. The components can be represented as data from a light capture device. Also, the method can include identifying subsets of light components, each subset of light components associated with one or more frequencies, identifying at a processor a time-domain component associated with a change in blood volume associated with the one or more surfaces of the organism, and extracting a physiological characteristic based on the time-domain component.

### CROSS-RELATED APPLICATIONS

This U.S. non-provisional patent application claims the benefit and priority to U.S. Provisional Patent Application No. 61/641,672 filed on May 2, 2012, which is incorporated by reference herein for all purposes.

### FIELD

Embodiments relate generally to the health field, and more specifically to a new and useful method for measuring detecting physiological characteristics, such as heart rate, of an organism.

### BACKGROUND

The heart rate of an individual can be associated with a wide variety of characteristics of the individual, such as health, fitness, interests, activity level, awareness, mood, engagement, etc. Simple to highly-sophisticated methods for measuring heart rate currently exist, from finding a pulse and counting beats over a period of time to coupling a subject to an EKG machine. However, each of these methods require contact with the individual, the former providing a significant distraction to the individual and the latter requiring expensive equipment.

Thus, there is a need to create a new and useful method for detecting physiological characteristics, such as heart rate, of an organism.

### BRIEF DESCRIPTION OF THE FIGURES

Various embodiments or examples (“examples”) of the invention are disclosed in the following detailed description and the accompanying drawings:

FIG. 1 is functional block diagram depicting an implementation of a physiological characteristic determinator, according to some embodiments;

FIGS. 2 to 3 depict various examples of implementing a physiological characteristic determinator, according to various embodiments;

FIGS. 4 to 6 depict various examples of determining physiological characteristics based on analysis of reflected light, according to various embodiments;

FIGS. 7 to 12 depict various applications using physiological characteristics based on analysis of reflected light, according to various embodiments; and

FIG. 13 illustrates an exemplary computing platform disposed in a computing device in accordance with various embodiments.

### DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

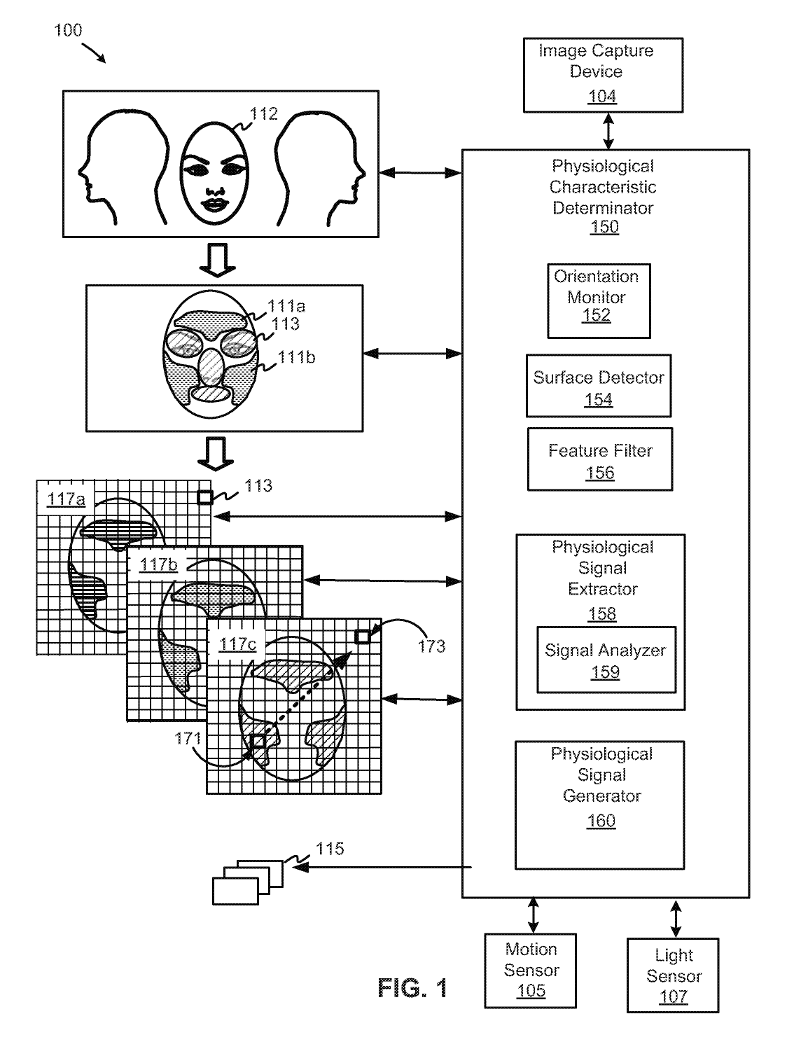

FIG. 1 is functional block diagram depicting an implementation of a physiological characteristic determinator, according to some embodiments. Diagram 100 depicts a physiological characteristic determinator 150 is coupled to a light capture device 104, which also can be an image capture device, such as a digital camera (e.g., video camera). As shown, physiological characteristic determinator 150 includes an orientation monitor 152, a surface detector 154, a feature filter 156, a physiological signal extractor 158, and a physiological signal generator 160. Surface detector 154 is configured to detect one or more surfaces associated with an organism, such as a person. As shown, surface detector 154 can use, for example, pattern recognition or machine vision, as described herein, to identify one or more portions of a face of the organism. As shown, surface detector 154 detects a forehead portion 111a and one or more cheek portions 111b. Feature filter 156 is configured to identify features 113 other than those associated with the one or more surfaces to filter data associated with pixels representing the features. For example, feature filter 156 can identify feature 113, such as the eyes, nose, and mouth to filter out related data associated with pixels representing the features. Thus, physiological characteristic determinator 150 processes certain face portions and “locks onto” those portions for analysis.

Orientation monitor 152 is configured to monitor orientation 112 of the face of the organism, and to detect a change in orientation in which at least one face portion is absent. For example, the organism may turn its head away, thereby removing a cheek portion from image capture device 104. In response, physiological characteristic determinator 150 can compensate for the absence of check portion, for example, by enlarging the surface areas of the face portions, by amplifying or weighting pixel values and/or light component magnitudes differently, or by increasing the resolution in which to process pixel data, just to name a few examples.

Physiological signal extractor 158 is configured to extract one or more signals including physiological information from subsets of light components captured by light capture device 104. For example, each subset of light components can be associated with one or more frequencies. According to some embodiments, physiological signal extractor 158 identifies a first subset of frequencies (e.g., a range of frequencies, including a single frequency) constituting green visible light, a second subset of frequencies constituting red visible light, and a third subset of frequencies constituting blue visible light. Other frequencies and wavelengths are possible, including those outside visible spectrum. As shown, a signal analyzer 159 of physiological signal extractor 158 is configured to analyze the pixel values or other color-related signal values 117a (e.g., green light), 117b (e.g., red light), and 117c (e.g., green light). For example, signal analyzer 159 can identify a time-domain component associated with a change in blood volume associated with the one or more surfaces of the organism. In some embodiments, physiological signal extractor 158 is configured to aggregate or average one or more AC signals from one or more pixels over one or more sets of pixels. Signal analyzer 159 can be configured to extracting a physiological characteristic based on, for example, a time-domain component based on, for example, using Independent Component Analysis (“ICA”) and/or a Fourier Transform.

Physiological data signal generator 160 can be configured to generate a physiological data signal 115 representing one or more physiological characteristics. Examples of such physiological characteristics include a heart rate pulse wave rate, a heart rate variability (“HRV”), and a respiration rate, among others, in a non-invasive manner.

According to some embodiments, physiological characteristic determinator 150 can be coupled to a motion sensor, 104 such as an accelerometer or any other like device, to use motion data from the motion sensor to determine a subset of pixels in a set of pixels based on a predicted distance calculated from the motion data. For example, consider that pixel or group of pixels 171 are being analyzed in association with a face portion. Upon detecting a motion (of either the organism or the image capture device, or both) in which such motion with move face portion out from pixel or group of pixels 171. Surface detector 154 can be configured to, for example, detect motion of a portions of the face in a set of pixels 117c, which affects a subset of pixels 171 including a face portion from the one or more portions of the face. Surface detector 154 predicts a distance in which the face portion moves from the subset of pixels 171 and determines a next subset of pixels 173 in the set of pixels 117c based on the predicted distance. Then, reflected light associated with the next subset of pixels 173 can be used for analysis.

In some embodiments, physiological characteristic determinator 150 can be coupled to a light sensor 107. Signal analyzer 159 can be configured to compensate for a value of light received from the light sensor that indicates a non-conforming amount of light. For example, consider that the light source generating the light is a fluorescent light source that, for instance, provides for less than desirable amount of, for example, green light. Signal analyzer 159 can compensate, for example, by weighting values associated with either the green light (e.g., either higher) or other values associated with other subsets of light components, such as red and blue light (e.g., weight the blue and red light to decrease influence of red and blue light). Other compensation techniques are possible.

In some embodiments, physiological characteristic determinator 150, and a device in which it is disposed, can be in communication (e.g., wired or wirelessly) with a mobile device, such as a mobile phone or computing device. In some cases, such a mobile device, or any networked computing device (not shown) in communication with physiological characteristic determinator 150, can provide at least some of the structures and/or functions of any of the features described herein. As depicted in FIG. 1 and subsequent figures (or preceding figures), the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in FIG. 1 (or any figure) can represent one or more algorithms. Or, at least one of the elements can represent a portion of logic including a portion of hardware configured to provide constituent structures and/or functionalities.

For example, physiological characteristic determinator 150 and any of its one or more components, such as an orientation monitor 152, a surface detector 154, a feature filter 156, a physiological signal extractor 158, and a physiological signal generator 160, can be implemented in one or more computing devices (i.e., any video-producing device, such as mobile phone, a wearable computing device, such as UP® or a variant thereof), or any other mobile computing device, such as a wearable device or mobile phone (whether worn or carried), that include one or more processors configured to execute one or more algorithms in memory. Thus, at least some of the elements in FIG. 1 (or any figure) can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, physiological characteristic determinator 150 and any of its one or more components, such as an orientation monitor 152, a surface detector 154, a feature filter 156, a physiological signal extractor 158, and a physiological signal generator 160, can be implemented in one or more circuits. Thus, at least one of the elements in FIG. 1 (or any figure) can represent one or more components of hardware. Or, at least one of the elements can represent a portion of logic including a portion of circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

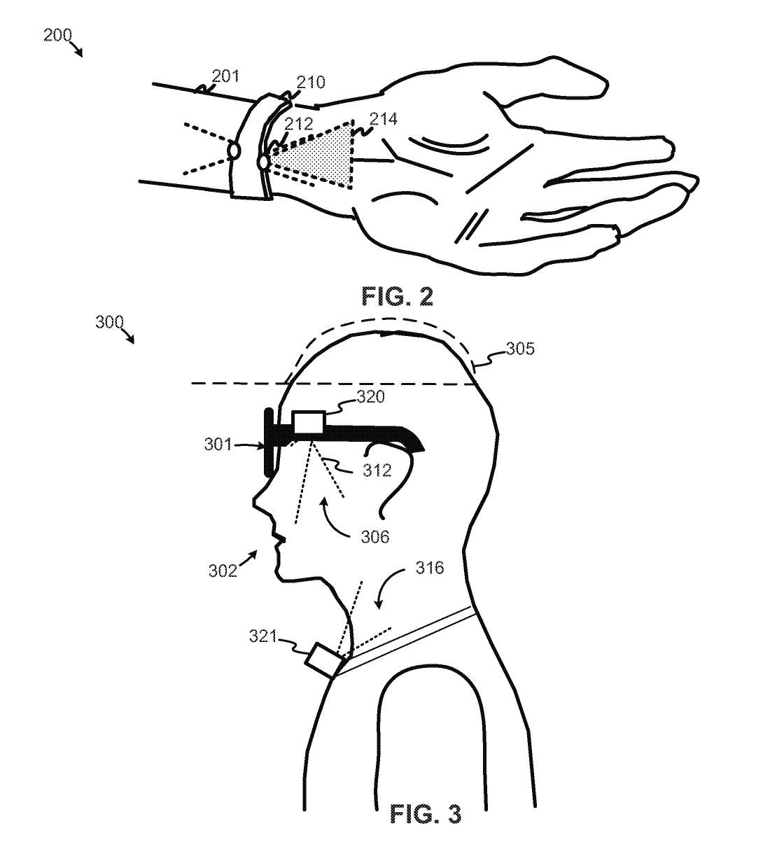

FIG. 2 depicts a wearable device 210 implementing a physiological characteristic determinator, according to some embodiments. The physiological characteristic determinator (not shown) is coupled to one or more light capture devices 212 to receive reflected light from surface portions 214. As shown, wearable device 210 is dispose on an organism's wrist and/or forearm, but can be located anywhere on a person. An example of a suitable wearable device, or a variant thereof, is described in U.S. patent application Ser. No. 13/454,040, which was filed on Apr. 23, 2012, which is incorporated herein by reference.

FIG. 3 depicts a wearable articles or apparel implementing a physiological characteristic determinator, according to some embodiments. Diagram 300 depicts physiological characteristic determinator disposed in a housing 320, which can couple to eyewear 301, a hat 305, or clothing of organism 302. The physiological characteristic determinator in housing 321 is coupled to clothing, such as a shirt collar, to receive light reflected by area 316, under which are relatively large volumes of blood that fluctuate and change over time. The physiological characteristic determinator in housing 320 is coupled to eyewear 301 to receive light 312 reflected by area 306, which is one of a number of face portions from which light is reflected.

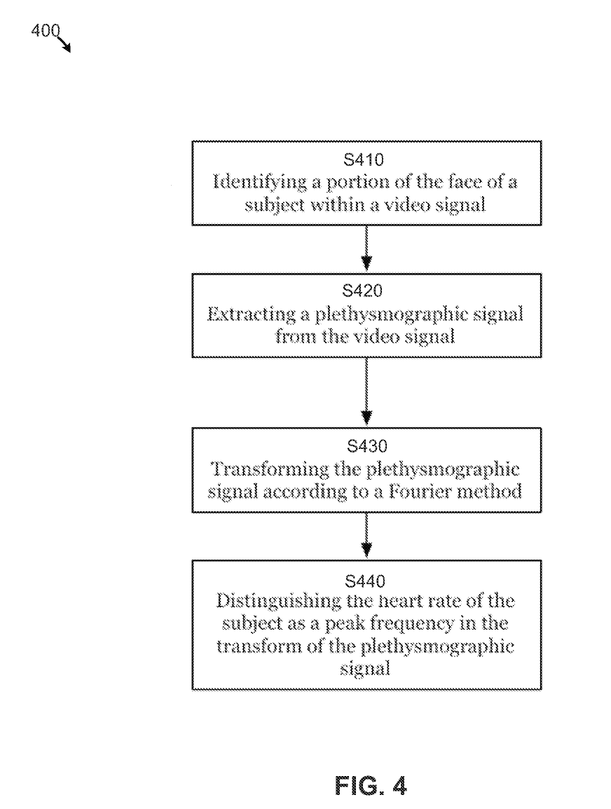

FIG. 4 depicts a flow for determining a physiological characteristic, according to some embodiments. Flow 400 provides for the determination of a physiological characteristic, such as the heart rate (HR) of a subject or organism. As show, flow 400 identifying a portion of the face of the subject within a video signal in Block S410; extracting or otherwise isolating a plethysmographic signal in the video signal through independent component analysis in Block S420; transforming the plethysmographic signal according to a Fourier method in Block S430; and identifying the heart rate of the subject as a peak frequency in the transform of the plethysmographic signal in Block S440.

The flow 400 functions to determine the HR of the subject through non-contact means, specifically by identifying fluctuations in the amount of blood in a portion of the body of the subject, as captured in a video signal, through component analysis of the video signal and isolation of a frequency peak in a Fourier transform of the video signal. The flow 400 can be implemented as an application or applet executing on an electronic device incorporating a camera, such as a cellular phone, smartphone, tablet, laptop computer, or desktop computer, wherein Blocks of the flow 400 are completed by the electronic device. Blocks of the flow 400 can additionally or alternatively be implemented by a remote server or network in communication with the electronic device. Alternatively, the flow 400 can be implemented as a service that is remotely accessible and that serves to determine the HR of a subject in an uploaded, linked, or live-feed video signal, though the flow 400 can be implemented in any other way. In the foregoing or any other variation, the video signal and pixel data and values generated therefrom are preferably a live feed from the camera in the electronic device, though the video signal can be preexisting, such as a video signal recorded previously with the camera, a video signal sent to the electronic device, or a video signal downloaded from a remote server, network, or website. Furthermore, the flow 400 can also include calculating the heart rate variability (HRV) of the subject and/or calculating the respiratory rate (RR) of the subject, or any other physiological characteristic, such as a pulse wave rate, a Meyer wave, etc.

In the example shown in FIG. 4, a variation of the flow 400 includes Block S405, which recites capturing red, green, and blue signals, for video signal, through a video camera including red, green, and blue color sensors. Block S405 can therefore function to capture data necessary to determine the HR of the subject without contact. The camera is preferably a digital camera (or optical sensor) arranged within an electronic device carried or commonly used by the subject, such as a smartphone, tablet, laptop or desktop computer, computer monitor, television, or gaming console.

The camera preferably operates at a known frame rate, such as fifteen or thirty frames per second, such that a time-domain component is associated with the video signal. The camera also preferably incorporates a plurality of color sensors, including distinct red, blue, and green color sensors, each of which generates a distinct red, blue, and green source signal, respectively. The color source signal from each color sensor is preferably in the form of an image for each frame recorded by the camera. Each color source signal from each frame can thus be fed into a postprocessor implementing other Blocks of the flow 400 to determine the HR, HRV, and/or RR of the subject. In some embodiments, a light capture device can be other than a camera, but can include any type of light (of any wavelength) receiving and/or detecting sensor.

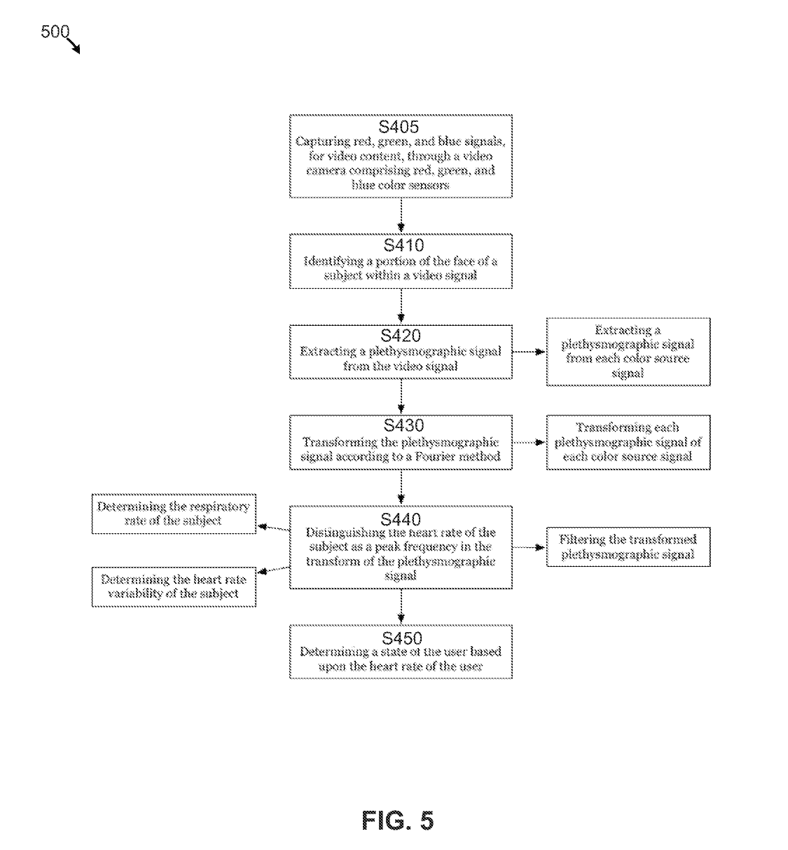

As shown in FIGS. 4 and 5, Block S410 of the flow 400 recites identifying a portion of the face of the subject within the video signal. Blood swelling in the face, particularly in the cheeks and forehead, occurs substantially synchronously with heartbeats. A plethysmographic signal can thus be extracted from images of a face captured and identified in a video feed. Block S410 preferably identifies the face of the subject because faces are not typically covered by garments or hair, which would otherwise obscure the plethysmographic signal. However, Block S410 can additionally or alternatively include identifying any other portion of the body of the subject, in the video signal, from which the plethysmographic signal can be extracted.

Block S410 preferably implements machine vision to identify the face in the video signal. In one variation, Block S410 used edge detection and template matching to isolate the face in the video signal. In another variation, Block S410 implements pattern recognition and machine learning to determine the presence and position of the face in video signal. This variation preferably incorporates supervised machine learning, wherein Block S410 accesses a set of training data that includes template images properly labeled as including or not including a face. A learning procedure can then transform the training data into generalized patterns to create a model that can subsequently be used to identify a face in video signals. However, in this variation, Block S410 can alternatively implement unsupervised learning (e.g., clustering) or semi-supervised learning in which at least some of the training data has not been labeled. In this variation, Block S410 can further implement feature extraction, principle component analysis (PCA), feature selection, or any other suitable technique to prune redundant or irrelevant features from the video signal. However, Block S410 can implement edge detection, gauging, clustering, pattern recognition, template matching, feature extraction, principle component analysis (PCA), feature selection, thresholding, positioning, or color analysis in any other way, or use any other type of machine learning or machine vision to identify the face of the subject in the video signal.

In Block S410, each frame of the video feed, and preferably each frame of each color source signal of the video feed, can be cropped of all image data excluding the face or a specific portion of the face of the subject. By removing all information in the video signal that is irrelevant to the plethysmographic signal, the amount of time required to calculate subject HR can be reduced.

As shown in FIG. 4, Block S420 of the flow 400 recites extracting a plethysmographic signal from the video signal. In the variation of the flow 400 in which the video signal includes red, green, and blue source signals, Block S420 preferably implements independent component analysis to identify a time-domain oscillating (AC) component, in at least one of the color source signals, that includes the plethysmographic signal attributed to blood volume changes in or under the skin of the portion of the face identified in Block S410. Block S420 preferably further isolates the AC component from a DC component of each source signal, wherein the DC component can be attributed to bulk absorption of the skin rather than blood swelling associated with a heartbeat. The plethysmographic signal isolated in the Block S420 therefore can define a time-domain AC signal of a portion of a face of the subject shown in a video signal. However, multiple color source-dependent plethysmographic signal can be extracted in Block S420, wherein each plethysmographic signal defines a time-domain AC signal of a portion of a face of the subject identified in a particular color source signal in the video feed. However, each plethysmographic signal can be extracted from the video signal in any other way in Block S420.

The plethysmographic signal that is extracted from the video signal in Block S420 is preferably an aggregate or averaged AC signal from a plurality of pixels associated with a portion of the face of the subject identified in the video signal, such as either or both cheeks or the forehead of the subject. By aggregating or averaging an AC signal from a plurality of pixels, errors and outliers in the plethysmographic signal can be minimized. Furthermore, multiple plethysmographic signals can be extracted in Block S420 for each of various regions of the face, such as each cheek and the forehead of the subject, as shown in FIG. 1. However, Block S420 can function in any other way and each plethysmographic signal can be extracted from the video signal according to any other method.

As shown in FIG. 4, Block S430 of the flow 400 recites transforming the plethysmographic signal according to a Fourier transform. Block S430 preferably converts the plethysmographic time-domain AC signal to a frequency-domain plot. In the variation of the flow 400 in which multiple plethysmographic signals are extracted, such as a plethysmographic signal for each of several color source signals and/or for each of several portions of the face of the user, Block S430 preferably includes transforming each of the plethysmographic signals separately to create a time-domain waveform of the AC component of each plethysmographic signal. Block S430 can additionally or alternatively include transforming the plethysmographic signal according to, for example, a Fast Fourier Transform (FFT) method, though Block S430 can function in any other way (e.g., using any other similar transform) and according to any other method.

As shown in FIG. 4, Block S440 of the flow 400 recites distinguishing the HR of the subject as a peak frequency in the transform of the plethysmographic signal. Because a human heart can beat at a rate in range from 40 beats per minute (e.g., highly-conditioned adult athlete at rest) to 200 beats for minute (e.g., highly-active child), Block S440 preferably functions by isolating a peak frequency within a range of 0.65 to 4 Hz, converting the peak frequency to a beats per minute value, and associating the beats per minute value with the HR of the subject.

In one variation of the flow 400, isolation of the peak frequency is limited to the anticipated frequency range that corresponds with an anticipated or possible HR range of the subject. In another variation of the flow 400, the frequency-domain waveform of Block S430 is filtered to remove waveform data outside of the range of 0.65 to 4 Hz. For example, in Block 140, the plethysmographic signal can be fed through a bandpass filter configured to remove or attenuate portions of the plethysmographic signal outside of the predefined frequency range. Generally, by filtering the frequency-domain waveform of Block S430, repeated variations in the video signal, such as color, brightness, or motion, falling outside of the range of anticipated HR values of the subject can be stripped from the plethysmographic signal and/or ignored. For example, alternating current (AC) power systems in the United States operate at approximately 60 Hz, which results in oscillations of AC lighting systems on the order of 60 Hz. Though this oscillation can be captured in the video signal and transformed in Block S430, this oscillation falls outside of the bounds of anticipated or possible HR values of the subject and can thus be filtered out or ignored without negatively impacting the calculated subject HR, at least in some embodiments.

In the variation of the flow 400 in which multiple plethysmographic signals are transformed in Block S430, Block S440 can include isolating the peak frequency in each of the transformed (e.g., frequency-domain) plethysmographic signals. The multiple peak frequencies can then be compared in Block S440, such as by removing outliers and averaging the remaining peak frequencies to calculate the HR of the subject. Particular color source signals can be more efficient or more accurate for estimating subject HR via the flow 400, and the particular transformed plethysmographic signals can be given greater weight when averaged with less accurate plethysmographic signal.

Alternatively, in the variation of the flow 400 in which multiple plethysmographic signals are transformed in Block S430, Block S440 can include combining the multiple transformed plethysmographic signals into a composite transformed plethysmographic signal, wherein a peak frequency is isolated in the composite transformed plethysmographic signal to estimate the HR of the subject. However, Block S440 can function in any other way and implement any other mechanisms.

In a variation of the flow 400 and as shown in FIG. 5, Block S440 can further include calculating the heart rate variability (HRV) of the subject through analysis of the transformed plethysmographic signal of Block S430. HRV can be associated with power spectral density, wherein a low frequency power component of the power spectral density waveform or the video signal or a color source signal thereof can reflect sympathetic and parasympathetic influences. Furthermore, the high frequency powers component of the power spectral density waveform can reflect parasympathetic influences. Therefore, in this variation, Block S440 preferably isolates sympathetic and parasympathetic influences on the heart through power spectral density analysis of the transformed plethysmographic signal to determine HRV of the subject.

In a variation of the flow 400 and as shown in FIG. 5, Block S440 can further include calculating the respiratory rate (RR) of the subject through analysis of the transformed plethysmographic signal of Block S430. In this variation, Block S440 preferably derives the RR of the subject through the high frequency powers component of the power spectral density, which is associated with respiration of the subject.

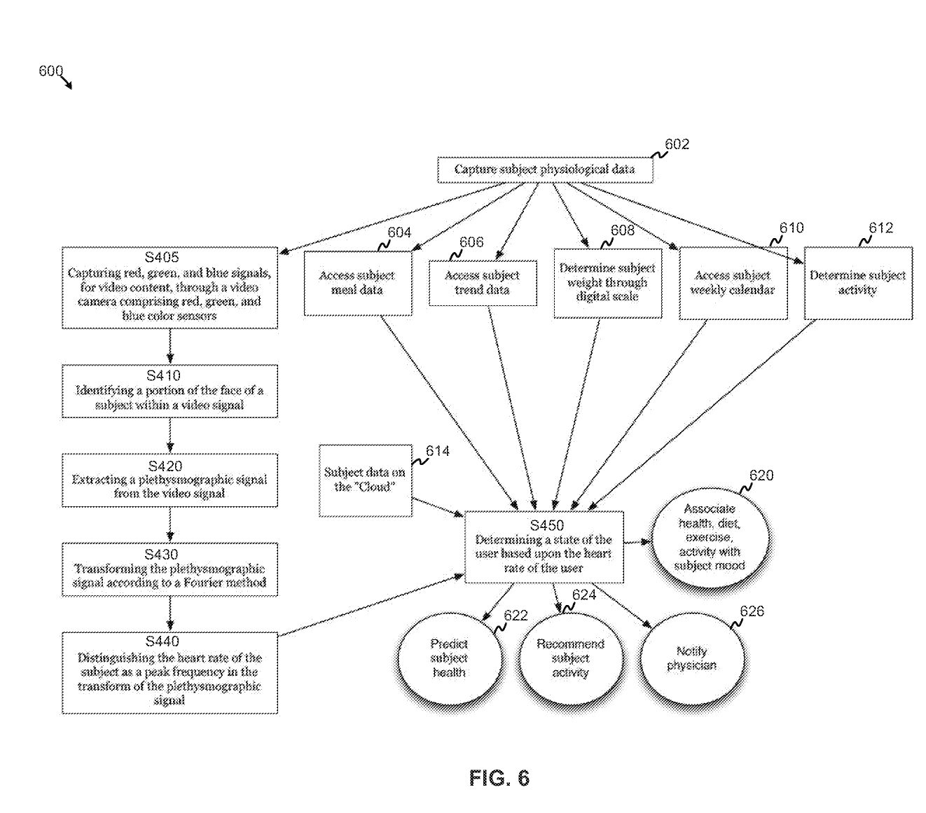

As shown in FIGS. 6 to 12, the flow 400 can further include Block S450, which recites determining a state of the user based upon the HR thereof. In Block S450, the HR, HRV, and/or RR of the subject is preferably augmented with an additional subject input, data from another sensor, data from an external network, data from a related service, or any other data or input. Block S450 therefore preferably provides additional functionality applicable to a particular field, application, or environment of the subject, such as described below.

FIG. 6 depicts an example of a varied flow, according to some embodiments. As shown in flow 600, flow 400 of FIG. 4 is a component of flow 600. At 602, physiological characteristic data of an organism can be captured and applied to further processes, such as computer programs or algorithms, to perform one or more of the following. At 604, nutrition and meal data can be accessed for application with the physiological data. At 606, trend and/or historic data can be used along with physiological data to determine whether any of actions 620 to 626 ought to be taken. Other information can be determined from 608 at which an organism's weight (i.e., fat amounts) is obtained. At 610, a subject's calendar data is accessed and an activity in which the subject is engaged is determined at 612 to determine whether any of actions 620 to 626 ought to be taken.

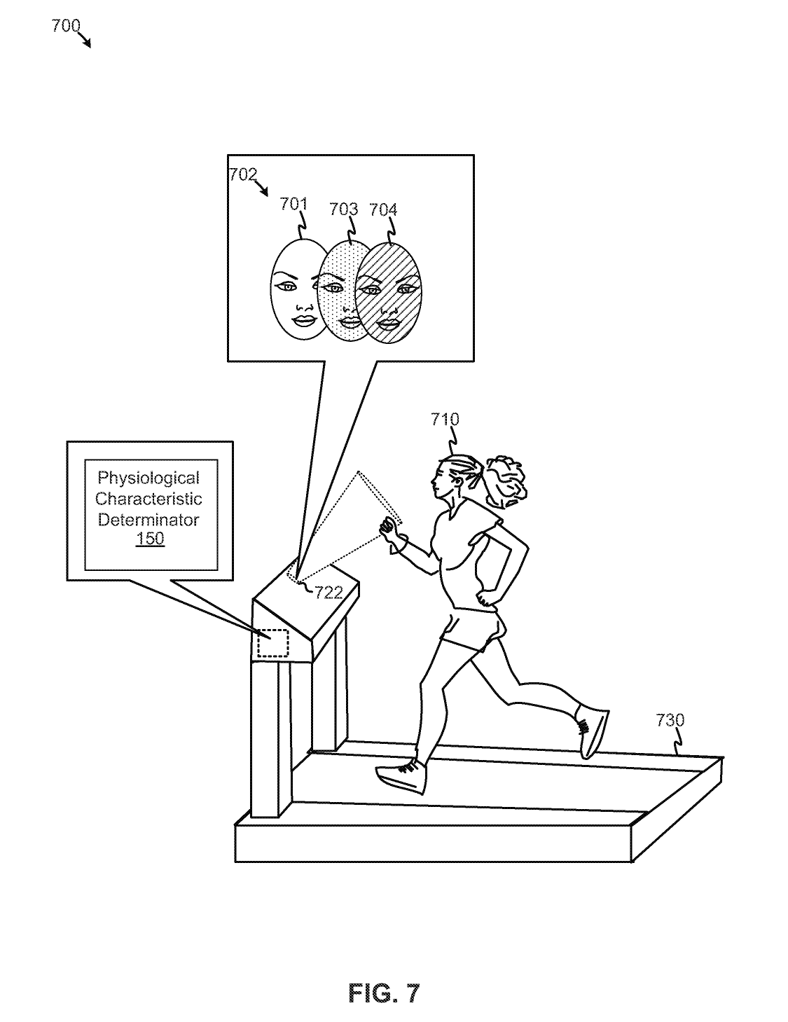

FIG. 7 depicts an example of a varied flow for obtaining physiological characteristics during non-incidental activities, such as exercise, according to some embodiments. As shown in FIG. 7, Block S450 can be varied and performed by, for example, a physiological characteristic determinator 150. In one example, the flow 400 is applied to exercise, wherein Block S450 includes determining a health-related metric of the subject. In this variation, physiological characteristic determinator 150 operates to monitor subject HR during exercise, such as by incorporating a camera and a processor (e.g., as part of physiological characteristic determinator 150) into an exercise machine (e.g., an elliptical or stationary bicycle). As shown, subject 710 is interacting with treadmill 730, whereby a light capture device 722 is configured to capture one or more subsets of light 702. In the example shown, reflected blue light 701 is captured, reflected red light 703 is captured, and reflected green light 704 is captured. This can be a simpler, more effective, and less expensive alternative to calculating subject HR that typically implements conductive pads incorporated into exercise equipment. Additionally or alternatively, the flow 400 can be implemented by a smartphone, tablet, computer, television, surveillance camera, or other electronic device arranged proximal or carried by the subject during exercise. Furthermore, the flow 400 can be used to estimate fitness metrics, such as recovery rate, which can be indicated by diminishing HR and RR of the subject over time following exercise or physical exertion.

By enabling a mobile device, such as a smartphone or tablet, to implement the flow 400, the subject can access any of the aforementioned calculations and generate other fitness-based metrics substantially on the fly and without sophisticated equipment. The flow 400, as applied to exercise, is preferably provided through a fitness application (“fitness app”) executing on the mobile device, wherein the app stores subject fitness metrics, plots subject progress, recommends activities or exercise routines, and or provides encouragement to the subject, such as through a digital fitness coach. The fitness app can also incorporate other functions, such as monitoring or receiving inputs pertaining to food consumption or determining subject activity based upon GPS or accelerometer data.

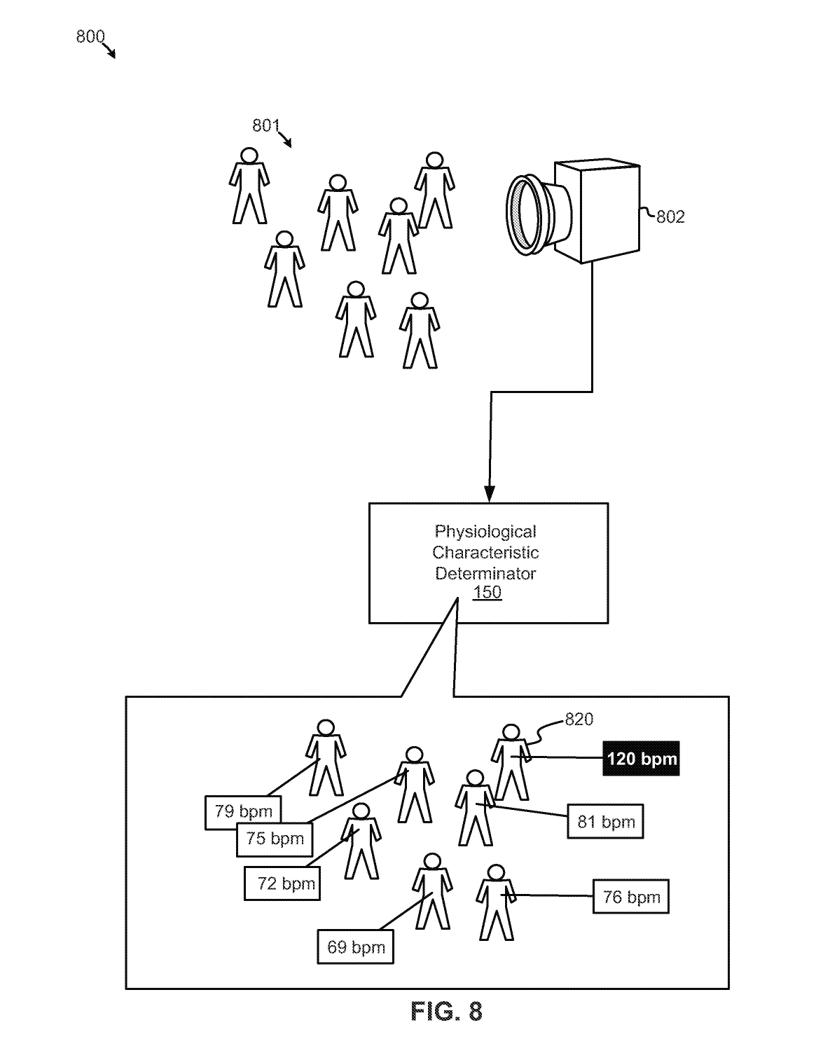

FIG. 8 depicts an example of a varied flow for obtaining physiological characteristics of a group of subjects, such as at an area in which security is paramount, according to some embodiments. As shown in FIG. 8, Block S450 can be varied and performed by, for example, a physiological characteristic determinator 150, which, in turn, performs flow 400 (or a portion thereof). As applied to surveillance and security, Block S450 includes anticipating a future crime-related action of the subject. In this variation, physiological characteristic determinator 150 (via light capture device 802) can capture elevated HRs of subject 820 in a group of subject 801, which can correlate with nervousness (e.g., higher-than normal heart rates) indicative of anticipation of a crime. For example, a retail store can implement the flow 400 to identify a plurality of faces of customers within the store and within view of the camera in Block S410, to determine the HRs of at least a portion of the customers in Block S440, and to predict a future crime based upon the HR of a particular customer that is significantly elevated above the HRs of other customers or that is significantly elevated above a threshold HR for low-crime-risk customers in Block S450. The flow 400 can be similarly used in airport security for prescreening purposes. For example, the flow 400 can be implemented in a 3D body scanner to check subject HR while undergoing a body scan. In another example, the flow 400 can be implemented within the cabin of a commercial airplane, such as to predict anticipate a future activity of an occupant. Alternatively, the flow 400 can be implemented in the form of a baby monitor to determine the status of a baby, such as if the baby is presently healthy as indicated by a HR within a proper range, is breathing properly, or is sleeping as indicated by a reduced RR.

The flow 400 can also be implemented in a crowd control setting. In one example, because HR can correlate with anxiety or anticipation of a future action, an officer or agent can engage a smartphone or other device incorporating a camera to monitor the HRs of various subjects in the crowd and thus anticipate a future action of a particular subject. Generally, the officer or agent can single out a subject in the crowd as a potential threat based upon a HR significantly greater than the HRs of proximal subjects. Alternatively, the officer or agent can adjust crowd control efforts (e.g., deployment of fencing, number of security personnel) based upon elevated HRs of subjects in the crowd or the average HR across a portion or all of the crowd. A microphone or other volume sensor can further corroborate the correlation between HR and anxiety for one or more subjects within the crowd.

According to some embodiments, flow 400 can be implemented by physiological characteristic determinator 150 in the service industry. For example, Block S450 can be configured to determine a mood of the subject. The flow 400 can calculate the HR of an employee of a call center (not shown) as an estimation of frustration with a customer, wherein a HR or correlated frustration level exceeding a threshold level can indicate need for a break or initiate transfer of a call to a supervisor or other employee. Alternatively, the HR of the customer can be monitored (e.g., remotely) to determine the same, such that customer dissatisfaction is limited by ensuring that his experience shifts (e.g., by speaking with a manager) before reaching a critical HR or correlated frustration level. In another alternative, a subject can be asked to look into the camera on his smartphone while on hold with a call center such that subjects with elevated HRs are given priority over subjects with less pending issues, as indicated by HR or correlated frustration level. In this variation, the flow 400 can be augmented by subject voice level, as captured by a microphone, wherein an elevated or rising voice volume reinforces estimated frustration level.

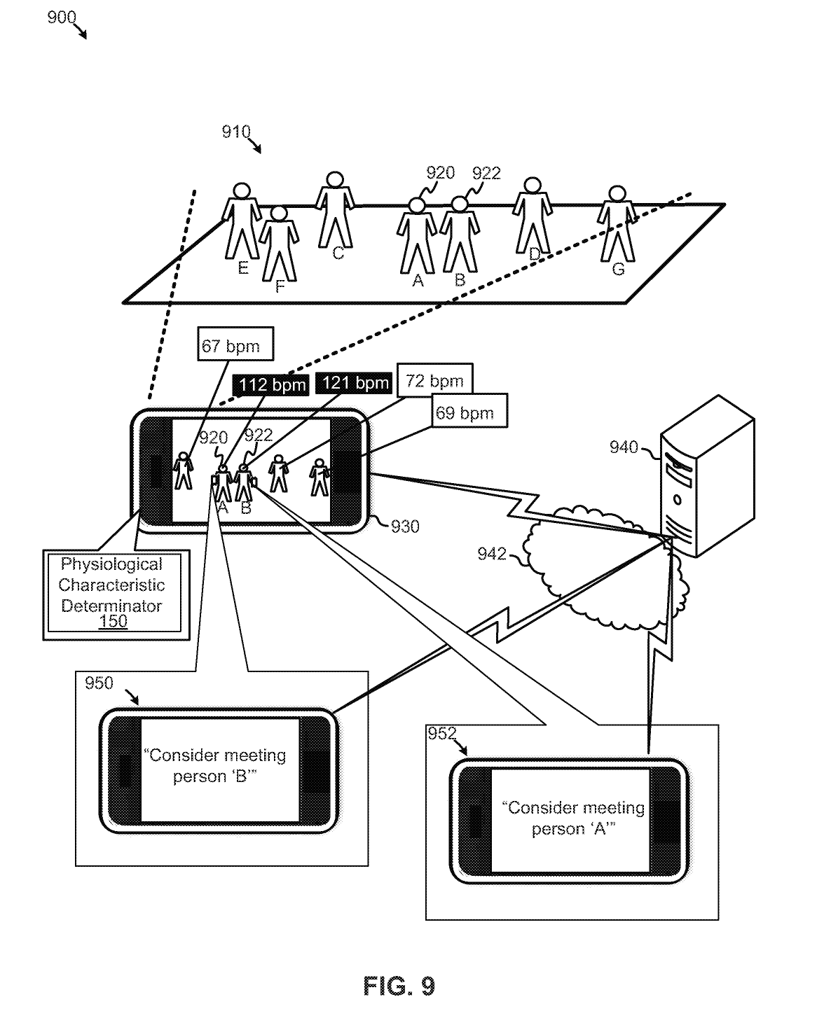

FIG. 9 depicts an example of a varied flow for obtaining physiological characteristics of a group of subjects to predict successful collaborations between two or more individuals, according to some embodiments. As shown in FIG. 9, Block S450 can be varied and performed by, for example, a physiological characteristic determinator 150, which, in turn, performs flow 400 (or a portion thereof). For example, consider a group of people 910 that includes persons A to G. As shown FIG. 9, the flow 400 can be applied to social environments, wherein Block S450 can be configured to determine interest, engagement, mood, activity level, or compatibility (e.g., to collaborate) of one or more subjects in a group of subjects 910. The flow 400 can be implemented in a live music concert setting to make on-the-fly suggestions, such as adjustment to tempo, volume, or a setlist to better maintain the HRs of subjects in the crowd within a desired HR range. For example, a relatively low average HR for a pop punk band can indicate that the band is playing at too slow a tempo, too softly, or has chosen songs not resonating with the audience. In another example, a relatively high average HR for diners at a fine restaurant can indicate that a band is playing too loud or too fast. Similarly a party host or partygoer can implement the flow 400, such as through a personal smartphone, to adjust the music, the activity, or the setting of the party to maintain partygoer interest, as correlated with subject HR, to within an acceptable or desired level.

The flow 400 can alternatively be used to guide introductions between two or more subjects based upon changes in HRs thereof. For example the flow 400 can be used to isolate two subjects (i.e., person (“A”) 920 and person (“B”) 922) in a crowd of subjects 910, wherein the two subjects experience elevated HRs when in proximity. This can indicate interest between the two subjects 920 and 922, and the flow 400 can further encourage at least one subject to make an introduction to the other subject. Computing device 930 can capture light reflected from subjects 920 and 922 to determine physiological characteristics using physiological characteristic determinator 150. As shown, subjects 920 and 922 have relatively elevated heart rates. Data from computing device 930 (e.g., a mobile phone device) can be transmitted via networks 942 to a remote computing device 940 that can operate as a social networking platform application.

Further to this example, flow 400 can thus be implemented as an ultra-local dating interface, at least in cooperation with remote computing device 940, wherein potential interest among multiple subjects 920 and 922 is corroborated with physical data, including the HRs of the subjects when in proximity. The flow 400 can also interface with a social or dating network, such as Facebook or Match.com, to ascertain the relationship status, interests, and other relevant information of at least one subject, which can better guide an introduction of the subjects. In this variation, the flow 400 (or a portion thereof) is preferably implemented as a “local dating app” executing on a smartphone, such as devices 950 and/or 952, such that a subject 920 or subject 920 can access the flow 400 without additional equipment beyond that available to the subject.

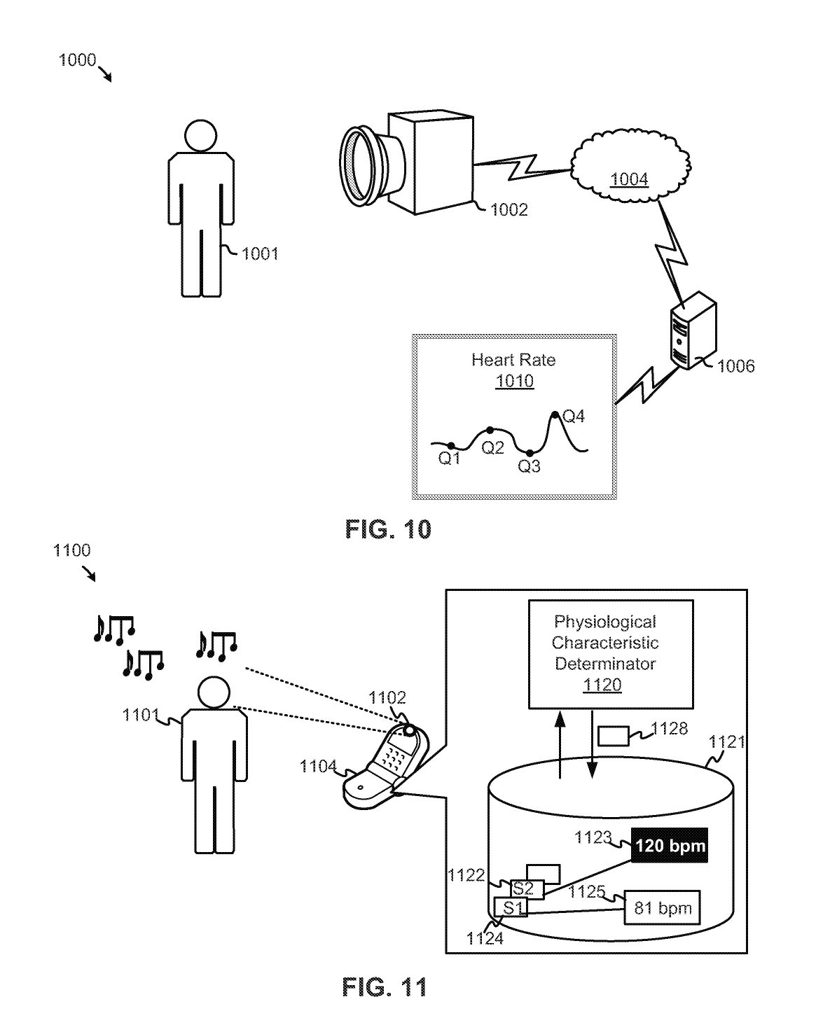

FIG. 10 depicts an example of a varied flow for obtaining physiological characteristics of one or more subjects responsive to stimuli for purposes of studying and/or testing one or more individuals, according to some embodiments. As shown in FIG. 10, Block S450 can be varied and performed by, for example, a physiological characteristic determinator (e.g., disposed in server computer 1006), which can perform flow 400 (or a portion thereof). For example, consider study participant 1001, whereby Block S450 includes determining a mental state or condition of the subject 1001. In one example implementation, the flow 400 is applied to mental studies, such as psychiatric evaluation interviews, wherein patient speech and body language indicators are augmented with the HR and RR of the patient. This can provide further insight into emotions, sources of anxiety, and other issues of the patient. The flow 400 can be implemented in real time, wherein video of a patient interview is captured by a camera 1002 arranged within an interview room and analyzed for patient HR substantially simultaneously. Alternatively, subject HR 1010 can be calculated post-hoc, wherein patient HR is extracted from an analog or digital video of the interview substantially after completion of the interview. Light data can be sent via network 1004 to a computing device 1006, which can operate to include a physiological characteristic determinator (not shown). In this second variation, videos recorded minutes, hours, days, weeks, or years prior can be analyzed for patient HR, including analog-format (e.g., tape cassette) video and digital-format video.

In another example implementation, the flow 400 is applied to subject testing to indicate subject satisfaction or subject frustration. For example, when a subject 1001 first purchases and downloads an app onto his smartphone, a forward-facing camera integral with the smartphone can capture initial subject reaction to the app, including subject HR, wherein an elevated subject HR indicates frustration or buyer's remorse and a steady subject HR indicates that the app meets subject expectations. The smartphone can also implement facial recognition or other machine vision techniques to capture a smile, frown, furrowed brow, etc. to further corroborate subject emotion following a purchase. In another example, a hardware company can study subject HR during assembly of a product, wherein an elevated HR during product assembly can indicate poor or confusing instructions, missing or mislabeled components, poor packaging, and/or a lower-than expected product quality. The flow 400 can therefore be implemented to qualify and quantify a subject experiences with a hardware or software product, particularly in situations in which a subject is unlikely or unable to provide feedback or in which a subject is typically unable to communicate specific problems or issues with a product.

In another example implementation, the flow 400 is implemented in the marketing and advertising space, wherein subject interest in a product, brand, advertisement, or advertising style is indicated by subject HR 1010 (shown in a computer display) as determined via the flow 400. For example, a camera 1002 integrated into a television or gaming console can capture sentiment or interest of one or more subjects while watching television. In the example shown, computing device 1006 is remote from light capture device 1002, but it does not need by in this or other examples. If the average HR of subjects watching a show on the television escalates during a romantic scene, advertisements and/or commercials presented to the subjects during the show can adjust to include ads for an upcoming romantic comedy, a romantic weekend getaway, or celebratory champagne. Alternatively, if the average HR of one or more subjects 1001 watching a show on the television escalates when food is shown, advertisements presented to the subjects during the show can adjust to include ads for fast-food restaurants or supermarkets. Generally, the flow 400 can be used to select ads more likely to resonate with a subject, wherein subject interest is associated with certain products or experiences based upon elevated subject HR.

In yet another example implementation, the flow 400 can be incorporated into polling services. For example, a public opinion poll for presidential candidates can ask voters to indicate a preferred candidate. A simultaneous elevation in HR of a voter can indicate a level of loyalty to or dislike for a particular candidate, which can provide more powerful information for political polling, such as devotion of a subset of voters to a particular candidate or the divisiveness of a particular candidate. Similarly, HRs of attendees of a debate can be monitored to ascertain topics that most resonate with a portion of demographic of the attendees or the nature of reactions to candidate responses.

In still another example implementation, the flow 400 is similarly employed to gauge public interest in new products, services, technologies, movies, etc. without directly polling the audience. For example, HRs of attendees of the Keynote presentation at an Apple® Worldwide Developers Conference (WWDC), in which a new product is revealed, can be aggregated as a means by which to gauge public interest without directly asking individuals for their opinions of the new product. However, the flow 400 can be applied to any other type of poll and in any other way.

FIG. 11 depicts another example of a varied flow for obtaining physiological characteristics of one or more subjects responsive to stimuli determine a level of interest or engagement of an organism to different stimuli, according to some embodiments. As shown in FIG. 11, Block S450 can be varied and performed by, for example, a physiological characteristic determinator 1120, which can perform flow 400 (or a portion thereof). In particular, Block S450 can be varied so that flow 400 can be applied to subject engagement, wherein Block S450 includes determining the level of engagement of a subject 1101 in a particular activity, such as listening to music, or advertisement. For example, for a subject 1101 watching an advertisement on a television, a computer, a tablet, or a smartphone, an elevated subject HR can indicate that the advertisement has piqued the interest of the subject 1101, whereas a substantially steady HR can indicate relative subject disinterest in the advertisement. Advertising content can then be adjusted until content is found that results in elevates the subject HR. Therefore, through the flow 400, ads can be targeted to the subject 1101 based upon subject data recorded and analyzed substantially in the background and without active subject input.

Similarly, a camera 1102 integrated into a laptop computer (or a mobile computing device 1104) can capture subject HR while the subject listens to music, and music selection can be adjusted to maintain the HR of the subject above or below a threshold HR based upon the environment or activity of the subject. Alternatively, rather simply than selecting a genre, artist, album, or song, the subject 1102 can additionally or alternatively select a target HR, wherein songs are selected according to a schedule that maintains the HR of the subject substantially near, above, or below the target HR. Furthermore, when the subject indicates a preference or dislike for a particular song or artist, the HR of the subject can suggest the degree to which the subject likes or dislikes the particular song or artist. For example, physiological characteristic determinator 1120 can generate preference data 1128 for storing in a repository 1121. Consider that subject 1101 has listened to a first song associated song file 1124 (“S1”) and to a second song associated song file 1122 (“S2”). Data 1125 representing a first heart beat and data 1123 representing a second heart beat are associated with song files 1124 and 1122, respectively. In some embodiments, physiological characteristic determinator 1120 determines that subject 1101 is more interested in song 2 rather than song 1 based on, at least heartbeat data 1123 and 1125.

In one example implementation, the flow 400 can be applied to a gaming environment, wherein the HR of a subject playing a game correlates with subject interest in the game. For example, a subject playing poker can experience an elevated HR given an above-average hand or before or after making a sizable bet, and the flow 400 can thus be employed to estimate a future action of the subject during such a game. In another example implementation, the flow 400 can be applied to a gaming console, wherein the HR of the subject correlates with the activity level of the subject while playing a game on the gaming console. Through the flow 400, subject HR can be used adjust game play mechanics to maintain, increase, or assuage game activity. However, the flow 400 can be applied to the field of engagement in any other way.

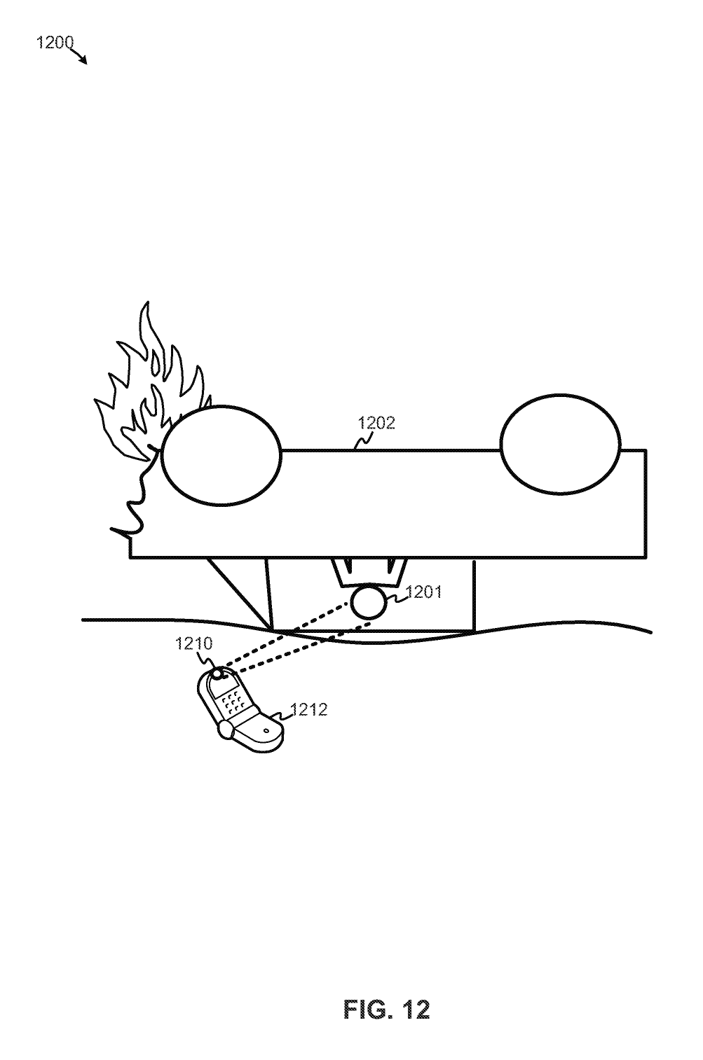

FIG. 12 depicts an example of a varied flow for obtaining physiological characteristics when the health or safety of an organism is at issue, according to some embodiments. Block S450 can be varied and performed by, for example, a physiological characteristic determinator (not shown) in cooperation with mobile device 1212, which can perform flow 400 (or a portion thereof). In particular, Block S450 can be varied to determine an immediate state of subject 1200 relating to the health and safety, or whether there exists risk factors affecting subject 1201. In one example implementation, the flow 400 is implemented by emergency personnel following an accident, wherein a paramedic or other user can evaluate the status of a victim 1201 (i.e. the subject) through non-contact means. Generally, in this example implementation, the flow 400 can be used to determine if the user is alive (e.g., has a heartbeat) and/or is breathing. This can be particularly useful if the victim is visible but not currently reachable, such as trapped within a vehicle 1202 of diagram 1200 or within a building, or if the victim can have suffered head, neck, or back trauma and contact with the victim should be minimized. The flow 400 can therefore also serve as a more reliable vital sign test than listening for a breath or checking an ulnar or carotid artery for a heartbeat, particularly in loud or dangerous environments, such as on a highway or battlefield.

In another example implementation, an older subject can set up cameras or light capture devices 1210 in a mobile device 1212 (or integrated in the interior of car 1202) at key locations within his car or home, wherein each camera checks the HR of the subject when the subject is within range. In this example implementation, a substantially low, high, or otherwise abnormal HR or RR can automatically alert a doctor or emergency staff of a potential health risk to the subject.

In yet another example implementation, the flow 400 provide safety and health warnings to a subject. For example, a subject engaging in yard work on a hot summer day can arrange a camera strategically within the yard, and the flow 400 can monitor subject HR and provide warnings if significant risk for sunstroke is calculated based upon changes in the HR, RR, perspiration rate, and/or activity level of the subject. The flow 400 can similarly be implemented through a camera integrated into a motor vehicle, wherein a lowered HR and/or RR of a driver of the vehicle can indicate that the driver is drowsy. This estimation can be corroborated by lowered eyelids or squinting, as captured by the camera. The subject can therefore by warned of elevated driving risk. Alternatively, the function of the vehicle can be automatically reduced to ameliorate the likelihood or severity of a pending accident.

In a further example implementation, the flow 400 can be implemented in a video game, such as Wii Tennis or Dance Dance Revolution (DDR), wherein the game can encourage subject activity up to a certain HR (i.e. based upon each individual subject) rather than based upon a preset maximum activity level. However, the flow 400 can be applied to safety in any other way.

Referring back to FIG. 6, another variation of Block S450, the flow 400 is applied to health. Block S450 can be configured to estimate a health factor of the subject. In one example implementation, the flow 400 is implemented in a plurality of electronic devices, such as a smartphone, tablet, and laptop computer, that communicate with each other to track the HR, HRV, and/or RR of the subject over time at 606 and without excessive equipment or affirmative action by the subject. For example, each instance of an activity at 612 in which the subject picks up his smartphone to make a call, check email, reply to a text message, read an article or e-book, or play Angry Birds, the smartphone can implement the flow 400 to calculate the HR, HRV, and/or RR of the subject. Furthermore, while the subject works in front of a computer during the day or relaxes in front of a television at night, the similar data can be obtained and aggregated into a personal health file of the subject. This data is preferably pushed, from each aforementioned device, to a remote server or network that stores, organizes, maintains, and/or evaluates the data. This data can then be made accessible to the subject, a physician or other medical staff, an insurance company, a teacher, an advisor, an employer, or another health-based app. Alternatively, this data can be added to previous data that is stored locally on the smartphone, on a local hard drive coupled to a wireless router, on a server at a health insurance company, at a server at a hospital, or on any other device at any other location.

HR, HRV, and RR, which can correlate with the health, wellness, and/or fitness of the subject, can thus be tracked over time at 606 and substantially in the background, thus increasing the amount of health-related data captured for a particular subject while decreasing the amount of positive action necessary to capture health-related data on the part of the subject, a medical professional, or other individual. Through the flow 400, health-related information can be recorded substantially automatically during normal, everyday actions already performed by a large subset of the population.

With such large amounts of HR, HRV, and/or RR data for the subject, health risks for the subject can be estimated at 622. In particular, trends in HR, HRV, and/or RR, such as at various times or during or after certain activities, can be determined at 612. In this variation, additional data falling outside of an expected value or trend can trigger warnings or recommendations for the subject. In a first example, if the subject is middle-aged and has a HR that remains substantially low and at the same rate throughout the week, but the subject engages occasionally in strenuous physical activity, the subject can be warned of increased risk of heart attack and encouraged to engage is light physical activity more frequently at 624. In a second example, if the HR of the subject is typically 65 bpm within five minutes of getting out of bed, but on a particular morning the HR of the subject does not reach 65 bpm until thirty minutes after rise, the subject can be warned of the likelihood of pending illness, which can automatically trigger confirmation a doctor visit at 626 or generation a list of foods that can boost the immune system of the subject. Trends can also show progress of the subject, such as improved HR recovery throughout the course of a training or exercise regimen.

In this variation, the flow 400 can also be used to correlate the effect of various inputs on the health, mood, emotion, and/or focus of the subject. In a first example, the subject can engage an app on his smartphone (e.g., The Eatery by Massive Health) to record a meal, snack, or drink. While inputting such data, a camera on the smartphone can capture the HR, HRV, and/or RR of the subject such that the meal, snack, or drink can be associated with measured physiological data. Overtime, this data can correlate certain foods correlate with certain feelings, mental or physical states, energy levels, or workflow at 620. In a second example, the subject can input an activity, such as by “checking in” (e.g., through a Foursquare app on a smartphone) to a location associated with a particular product or service. When shopping, watching a sporting event, drinking at a pub with friends, seeing a movie, or engaging in any other activity, the subject can engage his smartphone for any number of tasks, such as making a phone call or reading an email. When engaged by the user, the smartphone can also capture subject HR and then tag the activity, location, and/or individuals proximal the user with measured physiological data. Trend data at 606 can then be used to make recommendations to the subject, such as a recommendation to avoid a bar or certain individuals because physiological data indicates greater anxiety or stress when proximal the bar or the certain individuals. Alternatively, an elevated HR of the subject while performing a certain activity can indicate engagement in and/or enjoyment of the activity, and the subject can subsequently be encouraged to join friends who are currently performing the activity. Generally, at 610, social alerts can be presented to the subject and can be controlled (and scheduled), at least in part, by the health effect of the activity on the subject.

In another example implementation, the flow 400 can measure the HR of the subject who is a fetus. For example, the microphone integral with a smartphone can be held over a woman's abdomen to record the heart beats of the mother and the child. Simultaneously, the camera of the smartphone can be used to determine the HR of the mother via the flow 400, wherein the HR of the woman can then be removed from the combined mother-fetus heart beats to distinguish heart beats and the HR of the fetus alone. This functionality can be provided through software (e.g., a “baby heart beat app”) operating on a standard smartphone rather than through specialized. Furthermore, a mother can use such an application at any time to capture the heartbeat of the fetus, rather than waiting to visit a hospital. This functionality can be useful in monitoring the health of the fetus, wherein quantitative data pertaining to the fetus can be obtained at any time, thus permitting potential complications to be caught early and reducing risk to the fetus and/or the mother. Fetus HR data can also be cumulative and assembled into trends, such as described above.

Generally, the flow 400 can be used to test for certain heart or health conditions without substantial or specialized equipment. For example, a victim of a recent heart attack can use nothing more than a smartphone with integral camera to check for heart arrhythmia. In another example, the subject can test for risk of cardiac arrest based upon HRV. Recommendations can also be made to the subject, such as based upon trend data, to reduce subject risk of heart attack. However, the flow 400 can be used in any other way to achieve any other desired function.

Further, flow 400 can be applied as a daily routine assistant. Block S450 can be configured to include generating a suggestion to improve the physical, mental, or emotional health of the subject substantially in real time. In one example implementation, the flow 400 is applied to food, exercise, and/or caffeine reminders. For example, if the subject HR has fallen below a threshold, the subject can be encouraged to eat. Based upon trends, past subject data, subject location, subject diet, or subject likes and dislikes, the type or content of a meal can also be suggested to the subject. Also, if the subject HR is trending downward, such as following a meal, a recommendation for coffee can be provided to the subject. A coffee shop can also be suggested, such as based upon proximity to the subject or if a friend is currently at the coffee shop. Furthermore, a certain coffee or other consumable can also be suggested, such as based upon subject diet, subject preferences, or third-party recommendations, such as sourced from Yelp. The flow 400 can thus function to provide suggestions to maintain a energy level and/or a caffeine level of the subject. The flow 400 can also provide “deep breath” reminders. For example, if the subject is composing an email during a period of elevated HR, the subject can be reminded to calm down and return to the email after a period of reflection. For example, strong language in an email can corroborate an estimated need for the subject to break from a task. Any of these recommendations can be provided through pop-up notifications on a smartphone, tablet, computer, or other electronic device, through an alarm, by adjusting a digital calendar, or by any other communication means or through any other device.

In another example implementation, the flow 400 is used to track sleep patterns. For example, a smartphone or tablet placed on a nightstand and pointed at the subject can capture subject HR and RR throughout the night. This data can be used to determine sleep state, such as to wake up the subject at an ideal time (e.g., outside of REM sleep). This data can alternatively be used to diagnose sleep apnea or other sleep disorders. Sleep patterns can also be correlated with other factors, such as HR before bed, stress level throughout the day (as indicated by elevated HR over a long period of time), dietary habits (as indicated through a food app or changes in subject HR or RR at key times throughout the day), subject weight or weight loss, daily activities, or any other factor or physiological metric. Recommendations for the subject can thus be made to improve the health, wellness, and fitness of the subject. For example, if the flow 400 determines that the subject sleeps better, such as with fewer interruptions or less snoring, on days in which the subject engages in light to moderate exercise, the flow 400 can include a suggestion that the subject forego an extended bike ride on the weekend (as noted in a calendar) in exchange for shorter rides during the week. However, any other sleep-associated recommendation can be presented to the subject.

The flow 400 can also be implemented through an electronic device configured to communicate with external sensors to provide daily routine assistance. For example, the electronic device can include a camera and a processor integrated into a bathroom vanity, wherein the HR, HRV, and RR of the subject is captured while the subject brushes his teeth, combs his hair, etc. A bathmat in the bathroom can include a pressure sensor configured to capture at 608 the weight of the subject, which can be transmitted to the electronic device. The weight, hygiene, and other action and physiological factors can thus all be captured in the background while a subject prepares for and/or ends a typical day. However, the flow 400 can function independently or in conjunction with any other method, device, or sensor to assist the subject in a daily routine.

Other applications of Block S450 are possible. For example, the flow 400 can be implemented in other applications, wherein Block S450 determines any other state of the subject. In a one example, the flow 400 can be used to calculate the HR of a dog, cat, or other pet. Animal HR can be correlated with a mood, need, or interest of the animal, and a pet owner can thus implement the flow 400 to further interpret animal communications. In this example, the flow 400 is preferably implemented through a “dog translator app” executing on a smartphone or other common electronic device such that the pet owner can access the HR of the animal without additional equipment. In this example, a user can engage the dog translator app to quantitatively gauge the response of a pet to certain words, such as “walk,” “run,” “hungry,” “thirsty,” “park,” or “car,” wherein a change in pet HR greater than a certain threshold can be indicative of a current desire of the pet. The inner ear, nose, lips, or other substantially hairless portions of the body of the animal can be analyzed to determine the HR of the animal in the event that blood volume fluctuations within the cheeks and forehead of the animal are substantially obscured by hair or fur.

In another example, the flow 400 can be used to determine mood, interest chemistry, etc. of one or more actors in a movie or television show. A user can point an electronic device implementing the flow 400 at a television to obtain an estimate of the HR of the actor(s) displayed therein. This can provide further insight into the character of the actor(s) and allow the user to understand the actor on a new, more personal level. However, the flow 400 can be used in any other way to provide any other functionality.

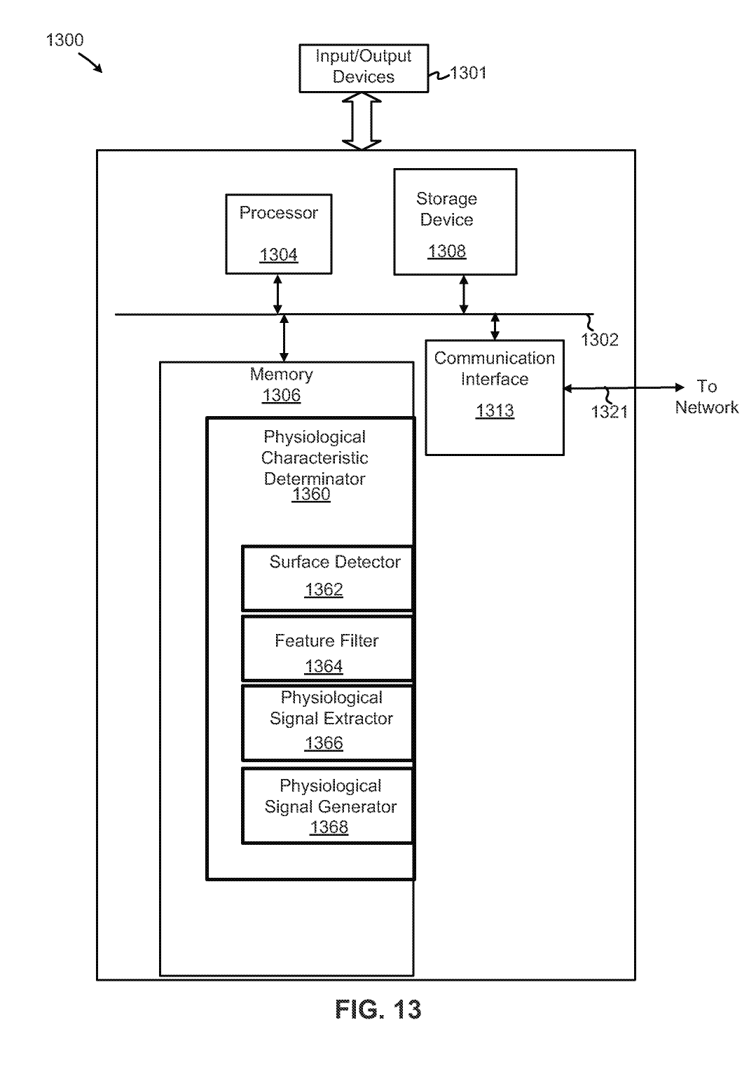

FIG. 13 illustrates an exemplary computing platform disposed in a computing device in accordance with various embodiments. In some examples, computing platform 1300 may be used to implement computer programs, applications, methods, processes, algorithms, or other software to perform the above-described techniques. Computing platform 1300 includes a bus 1302 or other communication mechanism for communicating information, which interconnects subsystems and devices, such as processor 1304, system memory 1306 (e.g., RAM, etc.), storage device 1308 (e.g., ROM, etc.), a communication interface 1313 (e.g., an Ethernet or wireless controller, a Bluetooth controller, etc.) to facilitate communications via a port on communication link 1321 to communicate, for example, with a computing device, including mobile computing and/or communication devices with processors. Processor 1304 can be implemented with one or more central processing units (“CPUs”), such as those manufactured by Intel® Corporation, or one or more virtual processors, as well as any combination of CPUs and virtual processors. Computing platform 1300 exchanges data representing inputs and outputs via input-and-output devices 1301, including, but not limited to, keyboards, mice, audio inputs (e.g., speech-to-text devices), user interfaces, displays, monitors, cursors, touch-sensitive displays, LCD or LED displays, and other I/O-related devices.

According to some examples, computing platform 1300 performs specific operations by processor 1304 executing one or more sequences of one or more instructions stored in system memory 1306, and computing platform 1300 can be implemented in a client-server arrangement, peer-to-peer arrangement, or as any mobile computing device, including smart phones and the like. Such instructions or data may be read into system memory 1306 from another computer readable medium, such as storage device 1308. In some examples, hard-wired circuitry may be used in place of or in combination with software instructions for implementation. Instructions may be embedded in software or firmware. The term “computer readable medium” refers to any tangible medium that participates in providing instructions to processor 1304 for execution. Such a medium may take many forms, including but not limited to, non-volatile media and volatile media. Non-volatile media includes, for example, optical or magnetic disks and the like. Volatile media includes dynamic memory, such as system memory 1306.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 1302 for transmitting a computer data signal.

In some examples, execution of the sequences of instructions may be performed by computing platform 1300. According to some examples, computing platform 1300 can be coupled by communication link 1321 (e.g., a wired network, such as LAN, PSTN, or any wireless network) to any other processor to perform the sequence of instructions in coordination with (or asynchronous to) one another. Computing platform 1300 may transmit and receive messages, data, and instructions, including program code (e.g., application code) through communication link 1321 and communication interface 1313. Received program code may be executed by processor 1304 as it is received, and/or stored in memory 1306 or other non-volatile storage for later execution.

In the example shown, system memory 1306 can include various modules that include executable instructions to implement functionalities described herein. In the example shown, system memory 1306 includes a Physiological Characteristic Determinator 1360 configured to implement the above-identified functionalities. Physiological Characteristic Determinator 1360 can include a surface detector 1362, a feature filter, a physiological signal extractor 1366, and a physiological signal generator 1368, each can be configured to provide one or more functions described herein.

The systems and methods of the preferred embodiment can be embodied and/or implemented at least in part as a machine configured to receive a computer-readable medium storing computer-readable instructions. The instructions are preferably executed by computer-executable components preferably integrated with a remote hospital, insurance, or health server, with hardware/firmware/software elements of a subject computer or mobile device, or any suitable combination thereof. Other systems and methods of the preferred embodiment can be embodied and/or implemented at least in part as a machine configured to receive a computer-readable medium storing computer-readable instructions. The instructions are preferably executed by computer-executable components preferably integrated by computer-executable components preferably integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component is preferably a processor but any suitable dedicated hardware device can (alternatively or additionally) execute the instructions.

As a person skilled in the art will recognize from the previous detailed description and from the figures and claims, modifications and changes can be made to the preferred embodiments of the invention without departing from the scope of this invention as defined in the following claims.

1. A method comprising:
detecting one or more surfaces associated with an organism;
receiving components of light reflected from the one or more surfaces of the organism, the components represented as data from a light capture device;
identifying subsets of light components, each subset of light components associated with one or more frequencies;
identifying at a processor a time-domain component associated with a change in blood volume associated with the one or more surfaces of the organism;
extracting a physiological characteristic based on the time-domain component;
causing transmission of data representing the physiological characteristic.

2. The method of claim 1, further comprising:
presenting on a display a graphical representation of the physiological characteristic,

3. The method of claim 1, wherein the physiological characteristic includes one or more of a heart rate, a pulse wave rate, a heart rate variability (“HRV”), and a respiration rate.

4. The method of claim 1, wherein detecting the one or more surfaces associated with an organism comprises:
identifying one or more portions of a face of the organism.

5. The method of claim 4, further comprising:
monitoring orientation of the face of the organism;
detecting a change in orientation in which at least one of the one or more portions of the face is absent; and
compensating for the absence of the at least one absent portion.

6. The method of claim 4, further comprising:
identifying features other than the one or more portions of the face; and
filtering data associated with pixels representing the features.

7. The method of claim 4, further comprising:
detecting motion of the one or more portions of the face in a set of pixels associated, a subset of pixels including a face portion from the one or more portions of the face;
predicting a distance in which the face portion moves from the subset of pixels;
determining a next subset of pixels in the set of pixels based on the predicted distance; and
receiving reflected light associated with the next subset of pixels.

8. The method of claim 4, further comprising:
detecting values of the components of light from a light source that generates the components of light;
determining at least one subset of the subsets of light components is associated with a value specifying a non-conforming amount of light; and
compensating for the non-conforming amount of the light.

9. The method of claim 8, wherein compensating for the non-conforming amount of the light comprises:
weighting values associated with either the subset or other values associated with other subsets of light components.

10. The method of claim 1, wherein identifying the subsets of light components comprises:
identifying a first subset of frequencies constituting green visible light, a second subset of frequencies constituting red visible light, and a third subset of frequencies constituting blue visible light.

11. The method of claim 1, further comprising:
extracting a plethysmographic signal.

12. The method of claim 1, wherein detecting the one or more surfaces associated with an organism comprises:
identifying one or more portions of a forearm including a wrist of the organism.

13. An apparatus comprising:
a light capture device; and
a processor configured to implement a physiological characteristic determinator, the physiological characteristic determinator comprising:
a surface detector configured to detect one or more surfaces associated with an organism;
a feature filter configured to identify features other than those associated with the one or more surfaces to filter data associated with pixels representing the features;
a physiological signal extractor configured to extract one or more physiological signals from subsets of light components captured by the light capture device, each subset of light components associated with one or more frequencies; and
a physiological data signal generator configured to generate a physiological data signal representing one or more physiological characteristics including a heart rate.

14. The apparatus of claim 13, further comprising:
a housing configured to couple to apparel associated with the organism.

15. The apparatus of claim 14, wherein the apparel comprises:
a hat including the housing,
wherein the light capture device is configured to capture light reflected from a face of the organism.

16. The apparatus of claim 14, wherein the apparel comprises:
eyewear including the housing,
wherein the light capture device is configured to capture light reflected from a face of the organism.

17. The apparatus of claim 14, wherein the apparel comprises:
a shirt having an opening for a neck of the organism,
wherein the light capture device is configured to capture light reflected from a neck of the organism.

18. The apparatus of claim 13, further comprising:
a band; and
a housing coupled to the band and including the light capture device,
wherein the light capture device is configured to capture light reflected from a wrist or forearm of the organism.

19. The apparatus of claim 13, further comprising:
a motion sensor,
wherein the processor is configured to use motion data from the motion sensor to determine a subset of pixels in a set of pixels based on a predicted distance calculated from the motion data.

20. The apparatus of claim 13, further comprising:
a light sensor,
wherein the processor is configured to compensate for a value of light received from the light sensor that indicates a non-conforming amount of light.
